(19) United States
(12) Patent Application Publication
(10) Pub. No.: US 2007/0270665 A1
(43) Pub. Date: Nov. 22, 2007

(54) Physiological Function Monitoring System

(76) Inventors: Chang-Ming Yang, Miaoli (TW); Chun-Mei Chou, Miaoli (TW)

Correspondence Address: BANGER SHIA, 204 CANYON CREEK, VICTORIA, TX, 77901, US

(21) Appl. No.: 11/744,209

(22) Filed: May 3, 2007

(30) Foreign Application Priority Data
May 5, 2006 (TW) 095115981

(51) Int. Cl. A61B 5/00

(52) U.S. Cl. 600/300; 600/586; 600/481; 600/323; 600/546; 600/547; 600/595; 600/529

(57) ABSTRACT

The invention provides a physiological function monitoring system for monitoring a first physiological function of a user. The physiological function monitoring system includes a first detecting device, N second detecting devices, a signal processing device, and a communicating device, N is a nature number. Furthermore, the physiological function monitoring system is applied in remote monitoring. In addition, the monitoring system is capable of detecting N second physiological functions of the user by the N detecting devices, to help making correct judgment on the first physiological function of the user.

### BACKGROUND OF THE INVENTION

1. Field of the Invention

The present invention relates to a portable physiological function monitoring system, and more particularly, to a portable physiological function monitoring system with high reliability.

2. Description of the Prior Art

With the development of life quality and medical care, the lifespan of human beings is significantly prolonged, and the elder population also increased. Facing the aged society, problems related to public welfare, medical technologies, and social security appeared, more and more aged people nowadays can not get sufficient care from family. Furthermore, because of the change in eating habits and lifestyle, the population with chronic diseases, such as hypertension, diabetes, gout, hyperlipemia syndrome, and heart diseases, has also increased quickly. Accordingly, most of the above-mentioned people need a real-time physiological function monitoring system to detect their physiological functions at anytime anywhere, to prevent the accidents from happening.

Recently, many researchers develop household physiological function monitoring system by combining sensing technologies and communicating technologies. What is more, related industries also begin to develop. The household physiological function monitoring system not only helps save the medical labor and resource which cost highly, but also helps people to notice abnormal health conditions as soon as possible. Accordingly, the household physiological function monitoring system is expected to be guarding the first line of health and medical care in the future.

With the development of physiological function detecting technologies and internet-related application technologies, remote physiological function monitoring will certainly be a new medication category in the coming future. According to the report by Industrial Economics & Knowledge Center (IEK), Taiwan, the scale of the remote medical care market in Taiwan, 2006 is measured at about 1,500 million NTD, and this number is expected to surge to 3,100 million NTD in 2010 with a compound annual growth rate of 19.5%. Obviously, remote medical care has become an important way to solve the problem of insufficient medical personnel and to enhance the quality of medical care. Moreover, through the application of new information technologies and equipments, the remote medical care system can satisfy the requirement of medical care for aged population and patients with chronic diseases, and reduce the times and cost of medication.

Furthermore, the value of the market for remote medical care in 2004 is about 4,400 million dollars, and this number is expected to rise to 7,600 million dollars in 2010. The development of a remote monitoring system for regularly monitoring a patient's physiological functions is one of the mature fields. Because the remote patient monitoring system has been covered by the insurance settlement in the United States, the scale of market reaches 54.5 million dollars (85% of the worldwide market) in 2003, and this number is expected to become 260 million dollars in 2010. Besides, the European market has been developed quickly, the compound growth rate will exceed 50%; whereas the Asian market will be the next sunrise market because of the increase of elder population.

As a result, consumers do have a need for the portable physiological monitoring system. However, in one way, the reliability of traditional portable physiological monitoring system is too low to provide dependable physiological information of a user to the monitoring personnel in the distal-end.

### SUMMARY OF THE INVENTION

Accordingly, an aspect of the invention is to provide a portable physiological function monitoring system, and more particularly, to a portable physiological function monitoring system with high reliability. Furthermore, the system of the invention can be remote-controlled and can be applied to assist monitoring personnel at the distal end to correctly determine a user's physiological function.

A portable physiological function monitoring system, according to a preferred embodiment of the invention, is used to monitor a first physiological function of a user. The physiological function monitoring system includes a first detecting device, N second detecting devices, a signal processing device, and a communicating device, wherein N is a nature number.

The first detecting device is used to detect the first physiological function, and generates a first signal in response to the detected first physiological function. The N second detecting devices are used to detect N second physiological functions of the user. Moreover, the N second detecting devices can generate N second signals in response to the detected N second physiological functions, and each of the N second signals is corresponding to one of the N second physiological functions respectively.

The signal processing device is connected to the first detecting device and the N second detecting devices, respectively, for receiving the first signal and the N second signals, and transmitting the first signal and the N second signals to a remote central monitoring station via a communicating device according to a first criterion. Furthermore, the communicating device is electrically connected to the signal processing device, for communicating with the remote central monitoring station.

A portable physiological function monitoring system, according to another preferred embodiment of the invention, is used to monitor a first physiological function of a user. The physiological function monitoring system includes a first detecting device, N second detecting devices, a signal processing device, and a communicating device, wherein N is a nature number.

The first detecting device is used to detect the first physiological function, and generates a first signal in response to the detected first physiological function. The N second detecting device is used to detect the first physiological function of the user respectively according to a driving signal, and generate N second signals in response to the detected first physiological function. Moreover, each of the N second signals is corresponding to the first physiological function respectively.

The signal processing device is connected to the first detecting device and the N second detecting devices respectively, for receiving the first signal and/or the N second signals. Moreover, the signal processing device can transmit the driving signal to at least one second detecting device to drive at least one second detecting device to detect the first physiological function. Additionally, the signal processing device can transmit the first signal and/or the N second signals to a remote central monitoring station via the communicating device according to a second criterion. The communicating device is electrically connected to the signal processing device, for communicating with the remote central monitoring station

The advantage and spirit of the invention may be understood by the following recitations together with the appended drawings.

### BRIEF DESCRIPTION OF THE APPENDED DRAWINGS

FIG. 1 shows an embodiment of the invention.

FIG. 2 shows a functional block of the physiological function monitoring system of an embodiment of the invention.

FIG. 3 shows a functional block of the physiological function monitoring system of an embodiment of the invention.

FIG. 4A shows an electrocardiogram of the prior art.

FIG. 4B shows a signal diagram of the electrocardiogram of FIG. 4A after filtration.

### DETAILED DESCRIPTION OF THE INVENTION

First of all, it should be noted that the term “physiological function” refers to the physiological functions, determined in the traditional medication, such as electrocardiogram, heartbeat, respiration, blood oxygenation level, body temperature, blood pressure, body resistance, . . . etc. Moreover, the term “physiological function” also refers to physiological functions, such as limb movement and amount of biological elements.

Furthermore, the above-mentioned term “biological elements” refers to any kind of molecule in human body, such as DNA, RNA, protein, antibody, and carbohydrate, . . . etc., and any kinds of trace elements, such as sodium, potassium, calcium, zinc, . . . etc., along with other suitable elements. The amount of these biological elements can be provided by medical device that is embedded in human body, such as biochips.

A preferred embodiment of the invention discloses a portable physiological function monitoring system for monitoring a first physiological function of a user.

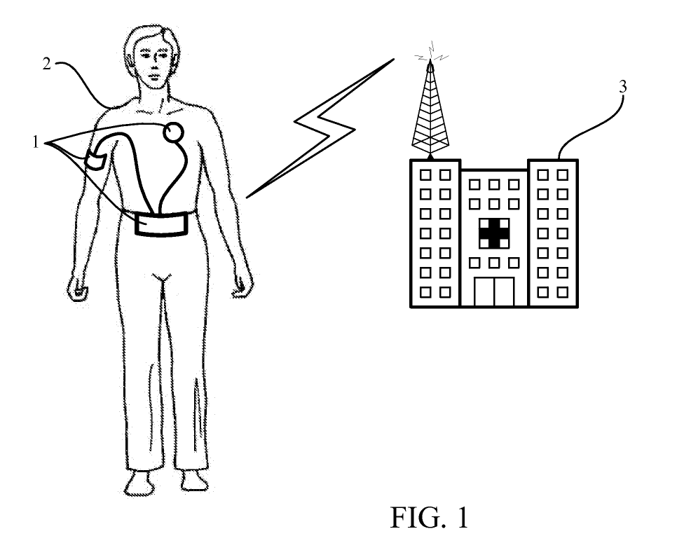

Please refer to FIG. 1, which shows an embodiment of the invention. As shown in FIG. 1, the monitoring system 1 can easily be carried by the user 2, and can be worn by the user 2 to monitor the first physiological function of the user 2 anytime and anywhere. Furthermore, the monitoring system 1 can transmit a signal related to the first physiological function to a remote central monitoring station 3, and receives the objective of remote monitor. In practice, the first physiological function can be, but not limited to, such as electrocardiogram, heartbeat, heart and lung sound, respiration (includes thoracic respiration, abdominal respiration, and thoracic/abdominal respiration), blood oxygenation level, body temperature, sweating level, blood pressure, electromyogram, body resistance, amount of biological elements, and limb movement. Additionally, the remote central monitoring station can be, for example, hospitals of different levels, medical care providers, and fire stations.

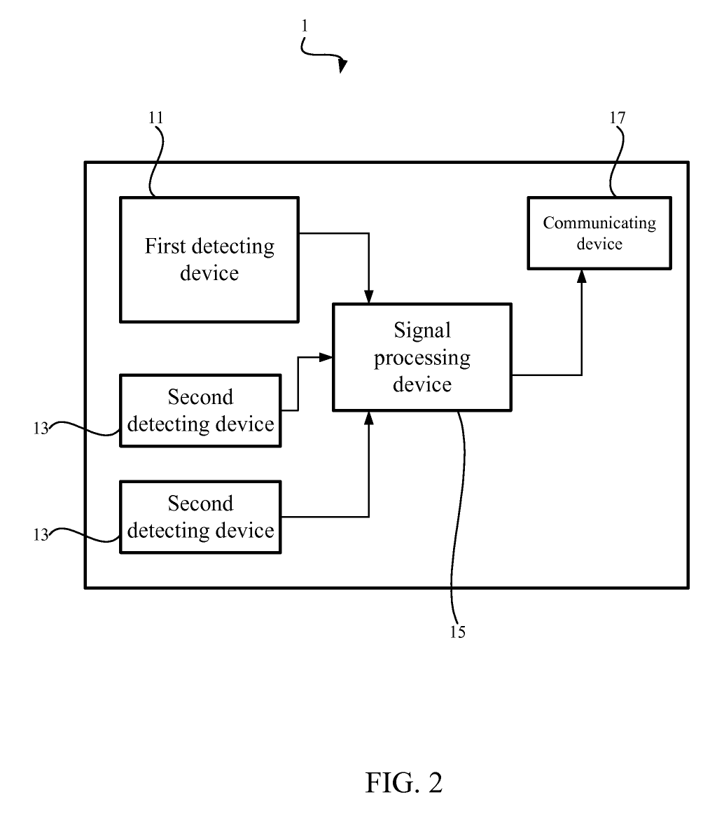

Please further refer to FIG. 2, which shows a functional block of the physiological function monitoring system of an embodiment of the invention. As shown in FIG. 2, the physiological function monitoring system 1 includes a first detecting device 11, two second detecting device 13, a signal processing device 15, and a communicating device 17. Please be noted that the amount of the second detecting devices 13 can be determined by practical requirements, and is, therefore, not limited to any number.

In practice, the first detecting device and the second detecting devices can be disposed on jewels, earrings, knee pads, hats, mobile phones, car chair cushions, helmets, shoes, sox, garments, bed sheets, and wheelchairs, or other suitable things.

In addition, the first detecting device 11 can contact the user's body, for detecting the first physiological function, and generating a first signal in response to the detected first physiological function. In practice, when the first physiological function includes heartbeat, respiration, or limb movement, the first detecting device includes at least a fabric-based gauge or an accelerometer.

These two second detecting device 13 can also contact the user's body, for detecting a second physiological functions respectively. Furthermore, these two second detecting device 13 can generate two second signals in response to the detected second physiological functions. Particularly, each of the second signals is correspond to one of the two detected second physiological functions.

In an embodiment, these two physiological functions can include at least heartbeat, respiration, or limb movement, and the second detecting device 13 includes at least a fabric-based gauge or an accelerometer, for detecting the limb movements, such as the movement of elbows, knees, and neck. The detection of the second physiological functions can assist the medical personnel at the distal-end to determine the reliability of the detected first physiological function, and help the monitoring personnel to give correct treatments.

In practice, the second physiological functions can be, but not limited to, electrocardiogram, heartbeat, heart and lung sound, respiration (includes thoracic respiration, abdominal respiration, and thoracic/abdominal respiration), blood oxygenation level, body temperature, sweating level, blood pressure, electromyogram, body resistance, amount of biological elements, and limb movements.

Furthermore, in the embodiment, the signal processing device 15 is connected to the first detecting device 11 and these two second detecting device 13, respectively, for receiving the first signal and these two second signals. The signal processing device 15 can further transmit the first signal and these two second signals to the remote central monitoring station (not shown) via the communicating device 17. In practice, the communicating device can include a learning mechanism, to automatically learn and remember the determining process of the first signal and the second signals to the remote central monitoring station. Therefore, when the signal processing device receives the same first signal and/or the second signals, the signal processing device can determine the signal itself without transmitting the signals to the remote central monitoring station.

Furthermore, the communicating device 17 is electrically connected to the signal processing device 15, and the communicating device 17 can communicate with the remote central monitoring station.

In practice, the signal processing device 15 and the first detecting device 11, along with these two second detecting device 13, can be connected through wired connection, such as electrical connection; or wireless connection, such as wireless RF connection.

In practice, the first criterion includes the signal processing device which determines if the first signal is abnormal, and when the signal processing device determines the first signal is abnormal, it will transmit the abnormal first signal and the two second signals to the remote central monitoring station via the communicating device 17.

In practice, the first criterion can also include the signal processing device 15 which transmits the first signal and these two second signals to the remote central monitoring station via the communicating device 17, when a pre-determined period or a pre-determined timing expires.

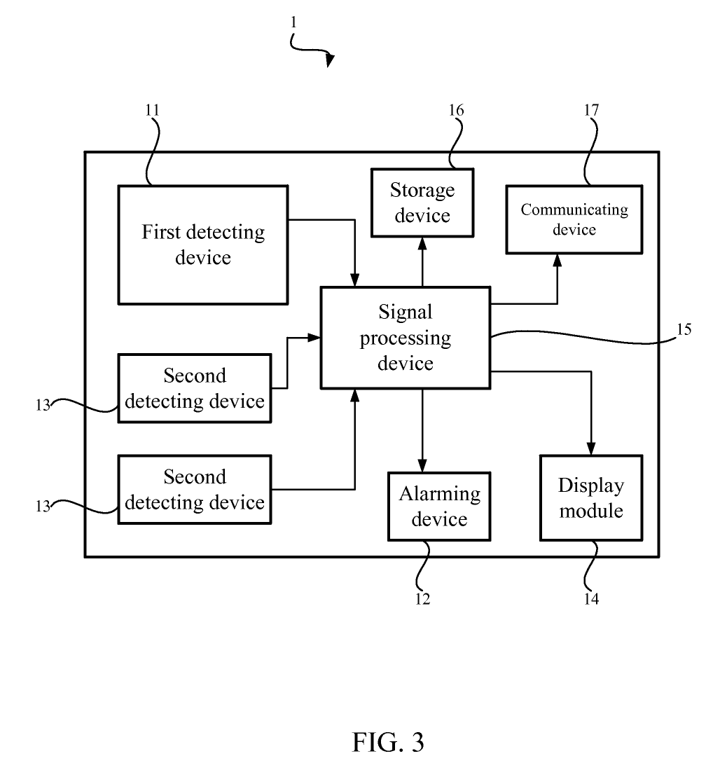

Please refer to FIG. 3, which shows a functional block of the physiological function monitoring system of an embodiment of the invention. As shown in FIG. 3, the physiological function monitoring system of the embodiment further includes an alarming device 12, a display module 14, and a storage device 16, besides the above-mentioned first detecting device 11, these two second detecting device 13, the signal processing device 15, and the communicating device 17.

The alarming device 12 is electrically connected to the signal processing device 15, and the signal processing device 15 determines if the first signal and these two second signals comply with a second criterion, the signal processing device 15 transmits a first triggering signal to the alarming device 12 to trigger the alarming device 12, and the alarming device 12 further generates a sound and/or a light and/or a text, and/or an image according to the first triggering signal. In practice, the second criterion can include the situation when the first signal is detected to be smaller or larger than a pre-defined value, the signal processing device 15 transmits the first triggering signal to trigger the alarming device 12.

In another embodiment, the alarming device can be further electrically connected to the communicating device. When the remote central monitoring station determines the first signal and the N second signals comply with a third criterion, the remote central monitoring station transmits a second triggering signal to the alarming device via the communicating device to trigger the alarming device, and the alarming device further generates the sound and/or the light and/or the text, and/or the image according to the second triggering signal. The third criterion includes the situation when the first signal is detected to be smaller or larger than a pre-defined value, the remote central monitoring station transmits the second triggering signal to trigger the alarming device.

Furthermore, the display module 14 is electrically connected to the signal processing device 15, for receiving and displaying the first signal and these two second signals. Therefore, the user can understand his or her physiological functions through the display module 14. Additionally, the display module 14 can also display the signals from the remote central monitoring station, and the user can understand the instructions or suggestions provided from the remote central monitoring station through the display module 14.

The storage device 16 is also connected to the signal processing device 15, for receiving the first signal and these two second signals and storing a first data corresponding to the first signal and two second data corresponding to the two second signals respectively.

In practice, the signal processing device, the communicating device, the alarming device, the storage device, the display module, and parts of the detecting devices can be integrated in the form of, for example, a mobile phone, a PDA, or a notebook.

Additionally, in practice, when the remote central monitoring station receives the signals, it can transmit a first confirming signal to the signal processing device via the communicating device. On the other hand, if the remote central monitoring station does not receive the signals, it can transmit a notice signal to the communicating device to warn the user. When the communicating device receives the notice signal, it can feed back a feedback signal to the remote central monitoring station. Moreover, if the remote central monitoring station does not receive the feedback signal, it can warn the user by other ways, such as by phone. The signal processing device can further give an alarm via the alarming device, to warn the user. Afterward, when the user notices the alarm, he or she can transmit a second confirming signal to the remote central monitoring station via the physiological function monitoring system. In practice, the user can communicate with the remote central monitoring station via phones, e-mails, and faxes.

In practice, when the user is a patient with dementia, the physiological function monitoring system of the invention can further transfer the signals detected to a receiver of a medical personnel, for assisting the medical personnel to control the user's physiological functions.

In practice, when the user feels unwell, he or she can transmit a mayday to the remote central monitoring station via the physiological function monitoring system of the invention.

In practice, the physiological function monitoring station can transmit a request signal to the physiological function monitoring system of the invention anytime to request the system to transmit the signals related to the physiological functions of the user.

For example, when the physiological function monitoring system is used to monitor a user with asthma, the first detecting device can be a gauge placed on the user's chest, for obtaining the respiration rate of the user by detecting the change of resistance between two points of the user's chest. Furthermore, because some users take abdominal respiration, one of the second detecting devices can also be a gauge, disposed at the abdominal position of the user. Other second detecting devices can also be a gauge placed on the elbows and knees of the user, for detecting the limbs movement of the user. Furthermore, one of the second detecting devices can be a body temperature detecting device, for measuring the user's body temperature, and the other one of the second detecting device can be a heartbeat detecting device, for detecting the user's heartbeat.

When the first signal, generated by the first detecting device, shows that the user's respiration is accelerated, the remote monitoring center can refer to the second signals that are generated by the second detecting devices, to determine the real condition of the user. For example, when the second signals show that the user's limbs are moving quickly, the body temperature is rising, and the heartbeat rate is increasing, the remote monitoring center can determine that the user is doing exercise but not breaking out. On the contrary, if the second detecting devices show the user's limbs are not moving quickly, and the body temperature is not getting higher, the remote monitoring center can determine that asthma is breaking out.

For another example, when the physiological function monitoring system is used to monitor a user with myocardial infarction, the first detecting device can be used to detect the hear function of the user. In addition, the second detecting devices can be the above-mentioned fabric-based gauge or an accelerometer, for detecting the limb movement of the user, especially the movement of the elbows of the user. When the first signal reveals a condition of arrhythmia or when the first signal can not be detected (such as when the first detecting device breakdown, or when the first detecting device disposed on a wrong position, or where there are too many noises around), the remote monitoring center can refer to the second signals generated by the second detecting device to determine the real condition of the user. For instance, because a patient often holds his or her chest when myocardial infarction breaks out, when the second signal reveals that the elbows of the user move toward the user's body at the same time (the fabric-based gauge can be elongated by the force, so as to detect the change of its resistance), the remote monitoring center can determine that the myocardial infarction is breaking out according to the second signals.

For another example, when the physiological function monitoring system is used to monitor the heart function of a user, the first detecting device can be used to detect the user's electrocardiogram. Besides the fabric-based gauge as mentioned before, the second detecting device can further include a microphone to detect the user's heart sound. Because the electrocardiogram easily contains noise when the user is doing exercise, the microphone can detect the heart sound, and help the remote central monitoring station to determine the user's heart condition. Furthermore, the strain gauge can help to determine if the user is doing exercise, and cause the increased heartbeats.

For yet another example, when the physiological function monitoring system is used to monitor the heart function of a user, the first detecting device can be used to obtaining the electrocardiogram of the user. Moreover, the second detecting devices can be a detecting device for sweating level. When the second detecting device measures no sweating level, it is then considered normal that the first detecting device can not detect the electrocardiogram, because the skin of the user is too dry. In practice, when the accelerometer or the strain gauge detected that the user is tacking a rest, the first detecting device is expected to detect the electrocardiogram. If the first detecting device can not detect the electrocardiogram, the remote central monitoring station can remind the user if he or she did not dispose the detecting devices at the right positions or confirm the user's condition.

In practice, the physiological function monitoring system can also be used to monitor a user's psychological condition in daily life, such as if the user is telling a lie. In the embodiment, the first detecting device can be used to detect the user's heartbeats. Moreover, the second detecting devices can be used to detect the user's sweating level, body temperature, and respiration. Because it is easy for nervous people to have accelerated heartbeats, we can determine whether the user is lying by monitoring the user's heartbeats. Moreover, we can make a more precise determination with the assistance of the second detecting devices to detect the body temperature and the change of respiration.

In practice, when the user is working, and the EKG can not be detect on the traditional position of the user's body, we can obtain the EKG signal from two points of the user's buttocks if the accelerometer or the gauge detected the user is sitting on a chair and his or her legs are not moving.

In practice, the above-mentioned accelerometer can be a multi-axial accelerometer or a strain gauge, for monitoring the user's (regular) limb movement, to assist the signal processing device 15 filtering the first signal (such as a signal of heartbeat or respiration) detected by the first detecting device 11. Moreover, in practice, the multi-axial accelerometer and the strain gauge can be used to detecting physiological functions such as heartbeat and respiration, to increase the signal of heartbeat or respiration detected by the first detecting device 11. Furthermore, in practice, the multi-axial accelerometer and the strain gauge can also be used to more accurately measure the direction of limb movement, and obtain further information.

In practice, because normally a person needs to take a rest for about 10 minutes before having his blood pressure measured, the second detecting device can be an accelerometer to help determine if the user has taken a rest for 10 minutes.

In practice, because normally a person needs to hang down his hands about 3 minutes for having his armpit temperature measured, the second detecting device can be an accelerometer or strain gauge to help determine if the user has hung down his hands for 3 minutes.

Obviously, the portable physiological function monitoring system can reach the objective of monitoring physiological functions of the user anytime and anywhere. Furthermore, the detection of the second physiological functions can assist to correctly determine the first physiological function, which decreases the possibility of wrongful determination, and increases the reliability of remote medical care.

Another preferred embodiment of the invention discloses a portable physiological function monitoring system, for monitoring a first physiological function, such as electrocardiogram, heartbeat, heart and lung sound, respiration (includes thoracic respiration, abdominal respiration, and thoracic/abdominal respiration), blood oxygenation level, body temperature, sweating level, blood pressure, electromyogram, body resistance, amount of biological elements, and limb movement of a user. The system includes a first detecting device, N second detecting devices, a signal processing device, and a communicating device, wherein N is a nature number.

The first detecting device is placed on the user's body, for detecting the first physiological function, and generating a first signal in response to the detected first physiological function. The N second detecting devices can also be placed on the user's body, for detecting the first physiological function of the user respectively according to a driving signal. The N second detecting devices can further generate N second signals in response to the detected first physiological function. In practice, when the first physiological function includes heartbeat, respiration, or limb movement, the first detecting device or the N second detecting devices include at least a fabric-based gauge or an accelerometer.

Additionally, the signal processing device is connected to the first detecting device and the N second detecting devices respectively, for receiving the first signal and/or the N second signals. The signal processing device selectively transmits the driving signal to at least one second detecting device and drives it to detect the first physiological function. Moreover, the signal processing device transmits the first signal and/or the N second signals to a remote central monitoring station via the communicating device according to a second criterion.

In practice, the signal processing device can be designed to have learning mechanism, to automatically learn and remember the determining process of the first signal and the second signals to the remote central monitoring station. Therefore, when the signal processing device receives the same first signal and/or the second signals, the signal processing device can determine the signal itself without transmitting the signals to the remote central monitoring station.

In practice, the first criterion includes the signal processing device determines if the first signal is abnormal, and when the signal processing device determines the first signal is abnormal, the processing device transmits the driving signal to the at least one second detecting device and makes it detect the first physiological function.

In practice, the second criterion includes the signal processing device which determines if the first signal is abnormal, and when the signal processing device determines the first signal to be abnormal, the processing device transmits the abnormal first signal and the N second signals to the remote central monitoring station via the communicating device. Furthermore, the second criterion can also include the signal processing device which transmits the first signal and the N second signals to the remote central monitoring station via the communicating device, when a pre-determined period or a pre-determined timing expired.

In an embodiment, the portable physiological function monitoring system further includes an alarming device, electrically connected to the signal processing device. Moreover, the signal processing device determines if the first signal and the N second signals comply with a third criterion, and if yes, the signal processing device transmits a first triggering signal to the alarming device to trigger the alarming device, and the alarming device further generates a sound and/or a light and/or a text, and/or an image in response to the first triggering signal. In practice, the third criterion includes the situation when the first signal and/or the N second signals are detected to be smaller or larger than a pre-defined value, the signal processing device then transmits the first triggering signal to trigger the alarming device.

In another embodiment, the alarming device is further electrically connected to the communicating device, and when the remote central monitoring station determines the first signal and the N second signals comply with a fourth criterion, the remote central monitoring station transmits a second triggering signal to the alarming device via the communicating device to trigger the alarming device, and the alarming device further generates the sound and/or the light and/or the text, and/or the image according to the second triggering signal. In practice, the fourth criterion includes the situation when the first signal is detected to be smaller or larger than a pre-defined value, the remote central monitoring station then transmits the second triggering signal to trigger the alarming device.

In an embodiment, the portable physiological function monitoring system can further include a display module, electrically connected to the signal processing device, for receiving and displaying the first signal and/or the N second signals.

In an embodiment, the portable physiological function monitoring system can further include a storage device, electrically connected to the signal processing device, for receiving the first signal and/or the N second signals, and storing a first data corresponding to the first signal and/or N second data corresponding to the N second signals respectively.

In practice, the first detecting device can be disposed in a pacifier for detecting the mouth temperature of a baby, and the second detecting device can be disposed on the armpits of the baby for detecting his or her armpit temperature. When the first detecting device can not detect the mouth temperature (for example, the baby spits out the pacifier), the second detecting device can be driven to detect the armpit temperature, to make sure of the body temperature.

In practice, the first detecting device can be disposed in an earphone to detect the ear temperature, and the second detecting device can be disposed on the armpits to detect the armpit temperature. When the first detecting device can not detect the ear temperature (for example, the user takes off the earphone), the second detecting device can be driven to detect the armpit temperature, to make sure of the body temperature.

In practice, the first detecting device can be an accelerometer, disposed on a suitable position of the user's body, for detecting if the user is falling down. Moreover, the second detecting can be a fabric-based strain gauge or a video camera, disposed on the user's feet or legs. When the signal processing device or the remote central monitoring station can not determine if the user is falling down based on the signal generated by the first detecting device, the second detecting device can be driven to help to confirm it.

In practice, we can obtain the EKG signal via any two points on the user's body, therefore, when a first detecting device, disposed on an underwear, can not detect the EKG signal, we can obtain the signal from a second detecting device disposed on the underpants, gloves, or a mobile phone.

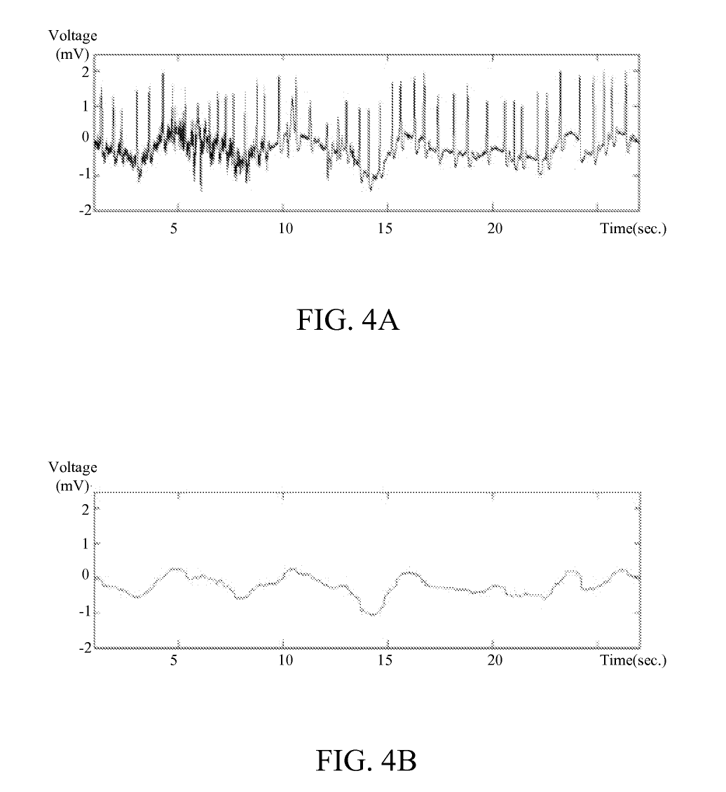

For example, when the physiological function monitoring system is used to monitor a user with asthma, the first detecting device can be a strain gauge placed on the user's chest, for obtaining the respiration rate of the user by detecting the change of resistance between two points of the user's chest. Moreover, one of the second detecting devices can include a plurality of electrodes to detect the electric signals generated by the movement of heart muscles, and then generate an electrocardiogram. Moreover, the electrocardiogram can be used to determine the user's respiration rate. (Please refer to FIG. 4A, which shows an electrocardiogram of the prior art. Because of the effect of respiration rate, the baseline of the electrocardiogram deviates up and down. Please refer to FIG. 4B, which shows a signal diagram of the electrocardiogram of FIG. 4A after filtration. As shown in FIG. 4B, after filtration, the signal diagram can represent the respiration rate. Accordingly, we can obtain the respiration rate of the user from the electrocardiogram.) Furthermore, one of the second detecting devices can be a microphone placed on a suitable place of the user's chest, for monitoring the user's chest sound, and generating a second signal in response to the chest sound. Additionally, one of the second detecting devices can be a heartbeat detecting device, for monitoring the user's heartbeats.

Obviously, the portable physiological function monitoring system can reach the objective of monitoring physiological functions of the user anytime and anywhere. Moreover, with the assistance of the second detecting devices on the first physiological detecting function, the operation of the monitoring system can be maintained even if the first detecting device or some of the second detecting devices failed or broke down. Furthermore, because the second detecting devices are optionally driven by the driving signal, the electronic power of the monitoring system can be effectively saved.

With the example and explanations above, the features and spirits of the invention are hopefully well described. Those skilled in the art will readily observe that numerous modifications and alterations of the device may be made while retaining the teaching of the invention. Accordingly, the above disclosure should be construed as limited only by the metes and bounds of the appended claims.

What is claimed is:

1. A portable physiological function monitoring system, for monitoring a first physiological function of a user, the portable physiological function monitoring system comprising:
a first detecting device, for detecting the first physiological function, and generating a first signal in response to the first physiological function detected;
N second detecting devices, for detecting N second physiological functions of the user, and generating N second signals in response to the detected N second physiological functions, wherein each of the N second signals is corresponding to one of the N second physiological functions respectively, N being a nature number;
a signal processing device, connected to the first detecting device and the N second detecting devices respectively, for receiving the first signal and the N second signals, and transmitting the first signal and the N second signals to a remote central monitoring station via a communicating device according to a first criterion; and
the communicating device, electrically connected to the signal processing device, for communicating with the remote central monitoring station.

2. The portable physiological function monitoring system of claim 1, wherein the first physiological function is one selected from the group consisting of: electrocardiogram, heartbeat, heart and lung sound, respiration, blood oxygenation level, body temperature, sweating level, blood pressure, electromyogram, body resistance, amount of biological elements, and limb movements.

3. The portable physiological function monitoring system of claim 2, wherein when the first physiological function comprises heartbeat, respiration, or limb movement, the first detecting device comprising at least a fabric-based gauge or an accelerometer.

4. The portable physiological function monitoring system of claim 1, wherein the N second physiological function comprises one selected from the group consisting of: electrocardiogram, heartbeat, heart and lung sound, respiration, blood oxygenation level, body temperature, sweating level, blood pressure, electromyogram, body resistance, amount of biological elements, and limb movement.

5. The portable physiological function monitoring system of claim 4, wherein when the second physiological function comprises heartbeat, respiration, or limb movements, the N second detecting devices comprising at least a fabric-based gauge or an accelerometer.

6. The portable physiological function monitoring system of claim 1, wherein the first criterion comprises the signal processing device determining if the first signal is abnormal, and when the signal processing device determines the first signal is abnormal, the processing device transmitting the abnormal first signal and the N second signals to the remote central monitoring station via the communicating device.

7. The portable physiological function monitoring system of claim 1, wherein the first criterion comprises the signal processing device transmitting the first signal and the N second signals to the remote central monitoring station via the communicating device, when a pre-determined period or a pre-determined timing expired.

8. The portable physiological function monitoring system of claim 1, further comprising an alarming device, electrically connected to the signal processing device, and the signal processing device determining if the first signal and the N second signals complying with a second criterion, the signal processing device transmitting a first triggering signal to the alarming device to trigger the alarming device, and the alarming device further generating a sound and/or a light and/or a text, and/or an image according to the first triggering signal.

9. The portable physiological function monitoring system of claim 8, wherein the second criterion comprises when the first signal is detected to be smaller or larger than a pre-defined value, the signal processing device transmitting the first triggering signal to trigger the alarming device.

10. The portable physiological function monitoring system of claim 8, wherein the alarming device is further electrically connected to the communicating device, and when the remote central monitoring station determines the first signal and the N second signals comply with a third criterion, the remote central monitoring station transmitting a second triggering signal to the alarming device via the communicating device to trigger the alarming device, and the alarming device further generating the sound and/or the light and/or the text, and/or the image according to the second triggering signal.

11. The portable physiological function monitoring system of claim 10, wherein the third criterion comprises when the first signal is detected to be smaller or larger than a pre-defined value, the remote central monitoring station transmitting the second triggering signal to trigger the alarming device.

12. The portable physiological function monitoring system of claim 1, further comprising a display module, electrically connected to the signal processing device, for receiving and displaying the first signal and the N second signals.

13. The portable physiological function monitoring system of claim 1, further comprising a storage device, electrically connected to the signal processing device, for receiving the first signal and N second signals and storing a first data corresponding to the first signal and N second data corresponding to the N second signals respectively.

14. A portable physiological function monitoring system, for monitoring a first physiological function of a user, the portable physiological function monitoring system comprising:
a first detecting device, for detecting the first physiological function, and generating a first signal in response to the first physiological function detected;
N second detecting devices, for detecting the first physiological function of the user respectively according to a driving signal, and generating N second signals in response to the detected first physiological function, wherein each of the N second signals is corresponding to the first physiological function respectively, N being a nature number;
a signal processing device, connected to the first detecting device and the N second detecting devices respectively, for receiving the first signal and/or the N second signals, and transmitting the driving signal to at least one second detecting device and driving it to detect the first physiological function, and the signal processing device transmitting the first signal and/or the N second signals to a remote central monitoring station via a communicating device according to a second criterion; and
the communicating device, electrically connected to the signal processing device, for communicating with the remote central monitoring station.

15. The portable physiological function monitoring system of claim 14, wherein the first physiological function is one selected from the group consisting of: electrocardiogram, heartbeat, heart and lung sound, respiration, blood oxygenation level, body temperature, sweating level, blood pressure, electromyogram, body resistance, amount of biological elements, and limb movements.

16. The portable physiological function monitoring system of claim 15, wherein when the first physiological function comprises heartbeat, respiration, or limb movements, the first detecting device or the N second detecting devices comprising at least a fabric-based gauge or an accelerometer.

17. The portable physiological function monitoring system of claim 14, wherein the first criterion comprises the signal processing device determining if the first signal is abnormal, and when the signal processing device determines the first signal is abnormal, the processing device transmitting the driving signal to the at least one second detecting device and driving it to detect the first physiological function.

18. The portable physiological function monitoring system of claim 14, wherein the second criterion comprises the signal processing device determining if the first signal is abnormal, and when the signal processing device determines the first signal is abnormal, the processing device transmitting the abnormal first signal and the N second signals to the remote central monitoring station via the communicating device.

19. The portable physiological function monitoring system of claim 14, wherein the second criterion comprises the signal processing device transmitting the first signal and the N second signals to the remote central monitoring station via the communicating device, when a pre-determined period or a pre-determined timing expired.

20. The portable physiological function monitoring system of claim 14, further comprising an alarming device, electrically connected to the signal processing device, and the signal processing device determining if the first signal and the N second signals complying with a third criterion, and if yes, the signal processing device transmitting a first triggering signal to the alarming device to trigger the alarming device, and the alarming device further generating a sound and/or a light and/or a text, and/or an image according to the first triggering signal.

21. The portable physiological function monitoring system of claim 20, wherein the third criterion comprises when the first signal and the N second signals are detected to be smaller or larger than a pre-defined value, the signal processing device transmitting the first triggering signal to trigger the alarming device.

22. The portable physiological function monitoring system of claim 20, wherein the alarming device is further electrically connected to the communicating device, and when the remote central monitoring station determines the first signal and the N second signals comply with a fourth criterion, the remote central monitoring station transmitting a second triggering signal to the alarming device via the communicating device to trigger the alarming device, and the alarming device further generating the sound and/or the light and/or the text, and/or the image according to the second triggering signal.

23. The portable physiological function monitoring system of claim 22, wherein the fourth criterion comprises when the first signal is detected to be smaller or larger than a pre-defined value, the remote central monitoring station transmitting the second triggering signal to trigger the alarming device.

24. The portable physiological function monitoring system of claim 14, further comprising a display module, electrically connected to the signal processing device, for receiving and displaying the first signal and/or the N second signals.

25. The portable physiological function monitoring system of claim 14, further comprising a storage device, electrically connected to the signal processing device, for receiving the first signal and/or the N second signals and storing a first data corresponding to the first signal and/or N second data corresponding to the N second signals respectively.
